(19) United States
(12) Patent Application Publication
(10) Pub. No.: US 2016/0155127 A1
(43) Pub. Date: Jun. 2, 2016

(54) SMART VENDING APPARATUS WITH AUTOMATED REGULATORY COMPLIANCE VERIFICATION AND METHOD OF USE

(71) Applicant: Alexander Lee Hartman, Monte Nido, CA (US)

(72) Inventor: Alexander Lee Hartman, Monte Nido, CA (US)

(21) Appl. No.: 14/557,343

(22) Filed: Dec. 1, 2014

(51) Int. Cl. G06Q 20/40; G06Q 20/20; G01N 33/487; H04N 5/76; G01N 33/497; G06Q 20/18; G06K 9/00

(52) U.S. Cl. CPC G06Q 20/40145; G06Q 20/18; G06Q 20/206; G06K 9/00228; G06K 9/00288; G06K 9/00087; G06K 9/00013; G06K 9/00604; G06K 9/00885; G06K 9/0061; H04N 5/76; G01N 33/4972; G01N 33/48792

(57) ABSTRACT

A smart vending machine and method of use for automated distribution of regulated products in compliance with all application statutory regulations of the relevant jurisdiction. Prospective consumers are positively identified by scanning and verifying the validity of a proffered government or specially issued identification and cross referencing associated pre-existing biometric information with a contemporary biometric analysis for congruency. For the distribution of regulated products requiring individualized authorization, permission granting authorities may confirm authorization remotely through secure portals.

### FIELD OF THE PRESENT DISCLOSURE

This disclosure relates generally to automated vending apparatuses and methods of use, and more particularly to smart vending apparatuses equipped with automated regulatory compliance verification features allowing for the automated distribution of statutorily regulated products to authorized individuals in compliance with all of the statutory regulations of the applicable jurisdiction.

### BACKGROUND OF THE RELATED ART

One of the most costly steps in the distribution channels of the various consumer products that are manufactured throughout the world is the last step; the retail step. Increasingly, vendors of all goods and services are looking for ways to reduce the cost of this last, retail step. For many, Internet websites provide the answer. Internet websites allow consumers to browse information regarding a product or service and order it directly from the manufacturer or distribution center thereby bypassing the need for costly retail locations and/or retail representatives. However, some products and services, by their nature and/or the nature by which consumers choose to purchase them, must be available in real-time and therefore cannot be ordered through Internet websites. Purveyors of such products must search for alternative, more immediate ways to reduce the cost of the last, retail step in the distribution channel.

One such class of products that must be available in real-time are individually packaged consumer edibles such as snacks and beverages. This is because individually packaged consumer edibles are usually purchased by a consumer to meet an immediate desire, and therefore, must be immediately available to satisfy the consumer. Traditionally, such products have been sold in small retail stores, sometimes called convenience stores, which are strategically located in areas with high consumer populations. As with other products, a convenience stores' overhead and labor are typically the largest cost adder in the entire distribution channel.

In the case of individually packaged consumer edibles, the cost of the final, retail level can be further reduced through the utilization of vending machines. Vending machines placed in areas with high consumer populations, can reliably distribute such products and collect payment without the need for the presence and supervision of a live retail representative. These both immediately meets consumers' desires and greatly reduces the overhead and labor costs of the final, retail step of the distribution channel.

Vending machines can be effectively used to sell a variety of other products as well so long as there is a need and/or desire for immediately. For example, consumer electronics and accessories that are most often sold through Internet websites are also sold through vending machines in airports, train stations, and other locations where consumers are travelling and occasionally have an immediate desire for a device or accessory.

Some products, despite enjoying strong consumer desires for immediate delivery, have not been successfully distributed through vending machines. This is typically because the federal, state, and/or local statutes that regulate the sale of such products are prohibitively complicated or require confirming the identity of the prospective purchaser, making compliance by traditional vending machines difficult not impossible. Examples of such regulated products are alcohol and tobacco products including e-cigarettes, prescription drugs, medical and recreational marijuana, and other age restricted products such as fireworks, spray paints, lottery tickets, firearm ammunition, certain solvents, and particular DVD's and computer games.

However, with use of modern technology, “smart” vending machines are capable of compliance by verifying an individual's identity and his or her eligibility to purchase a regulated item at a particular time and place. There exists need for smart vending machines that are equipped with modern technology to ensure compliance with all the relevant statutory regulations relevant to the sales of regulated products so that purveyors of such regulated products can use vending machines to reduce the cost of the final, retail step of distribution.

The sales of certain regulated products present some additional challenges as well. For example, purveyors of alcoholic beverages for immediate consumption are often prohibited from selling to individuals who are presently sufficiently intoxicated. In the past, automated compliance with such regulation was impossible; however, with the use modern technology “smart” vending machines can determine when an individual who is otherwise authorized to purchase an alcoholic beverage is temporarily ineligible due to the quantity of alcohol already consumed or the quantity already purchased within as set temporal period. This can be accomplished by assessing an individual's behavior, blood alcohol level, or the individual's immediate purchase history prior to authorizing a sale.

Another challenge that has prevented the use of vending machines for the distribution of regulated products is the perceived ease with which an unauthorized individual could convince an authorized individual to make purchases for him or her. While such unlawful schemes are possible even when purchasing regulated items from a retail representative, the perception exists that there is less risk of detection if the point of distribution is automated. However, with the use of modern technology, smart vending machines could tag all products with information making the purchaser's identity retroactively traceable. Such mechanism would create an ongoing risk of detection for authorized purchasers that may be tempted to make purchases for unauthorized individuals. Such smart vending machine that could tag regulated products with retroactively traceable purchaser identification would reduce or eliminate the perception that unauthorized individuals could obtain regulated products more easily through automated vending.

One further class of regulated products not currently distributed through vending machines are those products that require individualized permission to purchase such as prescription drugs or medical marijuana. Traditionally, distribution of such products have required a retail representative because processing a sale would necessarily include verifying that the prospective customer possessed a valid prescription or permission from a medical doctor or other proper permission granting authority. However, with the incorporation of modern technology, smart vending machines may be capable of verifying whether a prospective consumer is authorized to make a purchase of a regulated product directly with the permission granting authorities. There exists a need for smart vending machines so that the distribution of such individually regulated products can be automated so that the cost of distribution of these products can be reduced as well.

The present disclosure distinguishes over the related art providing heretofore unknown advantages as described in the following summary.

### SUMMARY OF THE INVENTION

The present disclosure relates to smart vending machines that through modern technology are capable of automated distribution of statutorily regulated products in compliance with all applicable statutory regulations of the relevant jurisdiction. Examples of such regulated products are alcohol and tobacco products including e-cigarettes, prescription drugs, medical and recreational marijuana, and other age restricted products such as fireworks, spray paints, lottery tickets, firearm ammunition, certain solvents, and particular DVD's and computer games.

There are many different general statutory restrictions and regulations to comply with when distributing regulated products, such as restrictions on the day of the week or the time of day during which such products are allowed to be sold. Temporal restrictions such as these can be easily programmed into the smart vending machine's programmable controller, preventing operation during restricted times. However, it is individualized regulations that present a greater challenge. Individualized regulations regarding the sales of regulated products are typically concerning the age of the prospective consumer. Positively verifying the identity of the prospective consumer becomes necessary to comply with such individualized regulations.

Traditionally, vending machines have not been capable of positively identifying a prospective consumer thereby requiring live retail representatives. When a retail representative sells a restricted product to a prospective consumer they essential perform two qualifying steps. First, they ask the prospective consumer for identification and determine whether the prospective consumer is eligible to make the desired purchase based on the information on the provided identification, and second, they perform a judgment as to whether the provided identification was, in fact, issued to the particular prospective consumer that proffered it. Unlike traditional vending machines, the presently disclosed apparatus can perform these steps similar to, if not superior than, a live retail representative.

When a prospective consumer approaches the presently disclosed apparatus, the smart vending machine begins the identification verification process by prompting the consumer to produce an accepted form of identification and to hold the identification in a location where the vending machine can “view” it. The apparatus is then capable of scanning an image of the proffered identification. In most embodiments, the apparatus may accept a driver's license, passport, or other form of government issued identification. Other accepted forms of identification may also include specially issued identification such as a medical marijuana card, a private club membership card, or a smart vending identification card issued to individuals after the completion of a prescreening process.

After viewing the identification, the smart vending apparatus may then identify key information such as any unique identifying numbers and/or other biographical information featured on the identification. Depending on the form of identification the apparatus is configured to accept, some embodiments may “view” the identification by scanning such key information from a magnetic stripe, RFID tag, transponder device, or microchip located on the identification.

The apparatus may then verify the authenticity of the proffered identification by comparing such key information against pre-existing information stored in a database, located either within the smart vending machine or in a remotely accessible database. If the key information scanned from the identification is congruent with the pre-existing information located in the database then the identification may be considered verified as valid and the apparatus may proceed to calculate the age of the prospective consumer according to biographical information associated with the proffered identification and/or utilize additional information from the identification to determine whether or not such prospective consumer is statutorily restricted from purchasing the particular selected regulated product.

In some embodiments, with the cooperation of the local jurisdictions, the remotely accessible database may be a government database. The smart vending machines may submit the collected information to the government database for verification as to whether the proffered identification is valid before determining whether such prospective consumer is authorized to purchase the particular regulated product pursuant to the key information extracted from the identification.

In other embodiments, the database may be a privately owned and operated database in which information regarding any government issued identification must be independently collected and entered into the database, or, as previously mentioned, such information may be collected through a prescreening process in which prospective consumers voluntarily submit such information to the database to register for eligibility to purchase restricted products through the smart vending machine. Whether located internal or external to the smart vending machine, this database may pertain to a singular apparatus or be networked with several smart vending machines.

Regardless of the configuration, the database is capable of verifying whether or not the identification presented by the prospective consumer is valid so that the smart vending machine can proceed to determine whether or not the individual to whom the identification was issued meets the statutory age restrictions or other individualized restriction for the particular regulated products.

After determining that the individual to which the proffered identification was issued is authorized to purchase a regulated product, the presently disclosed smart vending machine must also verify that the prospective consumer who presented the particular identification is, in fact, the individual to whom the identification was issued and pertains. To accomplish this, the prospective customer may then be instructed to face a camera so that an image of the prospective consumer's face can be accurately recorded. The image may then be compared the image of the individual to whom the identification was issued using facial recognition software. This may require the prospective customer to remove any hats, glasses, or other facial obstructions that may make a facial analysis more difficult. Further analysis such as an analysis of height and weight or other unique identifying features may be used as well to further verify the identity of the prospective consumer.

In embodiments in which the database is privately owned and operated and in which individuals are required to voluntarily submit to a prescreening to be eligible to purchase restricted products, other biometrics may be collected and used to verify the identity of prospective consumers as well. For example, prospective customers may submit to an iris scan, finger print, or handprint allowing the smart vending machine to have access to personal information not always possessed or willingly shared by the government. In other embodiments, prospective consumers may submit to a high quality facial scan rather than relying of facial recognition software to compare their image to the low quality image on their government issued identification. Still further embodiments may be a hybrid between the two, allowing individuals to submit further information to the database if they experience repeated issues or problems comparing their image to the low quality image on their government issued identification. This may also be necessary if the individual has experience as significant alteration in his or her features.

After verifying that both that the individual to whom the identification was issued is statutorily eligible to purchase the particular regulated product and that the prospective consumer who proffered such identification is, in fact, the individual to whom the identification was issued and pertains, the smart vending machine may allow the prospective consumer to proceed to pay for, and receive the particular selected regulated product in much the same way a traditional vending machine operates. However, because of the high level of identity verification required to make a purchase, such smart vending machines may also utilize more streamline purchasing methods such as monthly invoicing or automatically adding charges to a club account.

Still other embodiments may be configured to distribute regulated products that require individualized permission. Examples of such regulated products are prescription drugs, contact lenses, or medical marijuana. When attempting to automate the distribution of regulated products that require individualized permission to dispense, verification with the party authorized to grant such individualized permission such as a medical doctor is required.

Traditionally, a retail representative would review a medical prescription and perhaps call the doctor or other permission granting authority to verify the prescription's authenticity. While verifying a written prescription may be difficult for existing vending machines, the presently disclosed apparatus may be capable of allow the permission granting authority access to a secure portal to the smart vending machines database and directly communicate authorization for a particular individual. After such individualized permission is communicated to the presently disclosed apparatus, only verification that the prospective consumer is the particular individual to whom authorization as grant necessary to comply with the applicable statutory regulation.

Some embodiments are configured to comply with statutes that restrict the quantity of a particular regulated product a consumer could purchase within a specific temporal period or the frequency with which a consumer may make successive purchases. To comply with such restrictions, an individual's purchases may be recorded in the smart vending machine's database and reviewed when successive purchases are attempted.

Still other embodiments may attempt to inquire into a prospective consumer's current state of sobriety before selling such prospective consumer an alcoholic beverage. This may include a detection of eye nystagmus or other visual indication of intoxication when conducting a facial recognition scan, or in some embodiments, the smart vending machine may require a breathalyzer or other method of measuring blood alcohol before making a purchase.

One further feature of the presently disclosed apparatus that may be included in some embodiments of the present disclosure is the option of product purchase traceability. To discourage authorized individuals from purchasing regulated products for unauthorized individuals, some embodiments may record and preserve an audio and/or video record of the consummation of each transaction and mark each individually dispensed product with a unique tag associated with such recording, thereby allowing the administrator of the apparatus and/or law enforcement the ability to quickly retroactively identify the purchaser of each individually dispensed product by retrieving the relevant audio and/or video recording of the sales transaction.

The tag may be a bar code, QR code, or other marking that enables authorized personnel to quickly retrieve the portion of the transaction recording that features the identity of the individual to which regulated product was dispensed. In the event that law enforcement discovers unauthorized individuals in possession of a regulated product, this feature allows law enforcement to quickly identify the authorized individual who purchased a particular particular regulated product. Depending on the embodiment, law enforcement may be capable of accessing such audio and/or video recording directly, remotely through a secure portal, or through the cooperation of the administrator operating the smart vending machine. This feature can also be used to alert law enforcement when a potential customer presents invalid identification or identification that cannot be properly verified.

This disclosure teaches certain benefits in construction and use which give rise to the objectives described below:

A primary objective is to provide a smart vending apparatus with automated regulatory compliance verification and a method of use;

Another objective is to provide a smart vending apparatus with automated regulatory compliance verification including a method of use capable of confirming the identity of a prospective consumer to ensure regulatory compliance;

A further objective is to provide a smart vending apparatus with automated regulatory compliance verification and a method of use capable of verifying whether a prospective customer who is otherwise authorized to purchase a regulated products is temporarily ineligible due to present circumstances such as sobriety and/or the temporal duration since previous purchase;

A still further objective is to provide a smart vending apparatus with automated regulatory compliance verification and a method of use cable of independently verifying prospective consumers' individualized authorization to purchase a regulated product requiring individualized permission from a permission granting authority;

A yet still further objective is to provide a smart vending apparatus with automated regulatory compliance verification and a method of use capable of retroactively tracing purchaser identity;

Other features and advantages of the present invention will become apparent from the following more detailed description, taken in conjunction with the accompanying drawings, which illustrate, by way of example, the principles of the presently described apparatus and method of use.

### BRIEF DESCRIPTION OF THE DRAWING(S)

The accompanying drawings are diagrams that illustrate various exemplary implementations and are part of the specification. The illustrated implementations are proffered for purposes of example, not for purposes of limitation. Illustrated elements and steps will be designated by numbers. Once designated, an element or step will be identified by the identical number throughout. Illustrated in the accompanying diagram drawings is at least one of the best mode embodiments of the present disclosure. In such drawings:

FIG. 1 is a perspective view perspective view of an exemplary embodiment of the presently disclosed apparatus;

FIG. 2 is a perspective view perspective view of an exemplary embodiment of the presently disclosed apparatus featuring an exemplar prospective consumer preparing to commence the purchasing method of use;

FIG. 3 is a is a breakaway perspective view perspective view of an exemplary embodiment of the presently disclosed apparatus featuring an exemplar prospective consumer utilizing an identity verification feature.

FIG. 4 is a is a perspective view perspective view of an exemplary embodiment of the presently disclosed apparatus featuring an exemplar prospective consumer utilizing a documentation identify feature.

### DETAILED DESCRIPTION OF DRAWINGS OF AN EXEMPLARY EMBODIMENT

The above described drawings illustrate an exemplary embodiment of an apparatus in at least one of its preferred, best mode embodiments, which is further defined in detail in the following description. Those having ordinary skill in the art may be able to make alterations and modifications to what is described herein without departing from the spirit and scope of the disclosure. Therefore, it must be understood that what is illustrated is set forth only for the purposes of example, and that it should not be taken as a limitation of the scope of the present apparatus or its method of use.

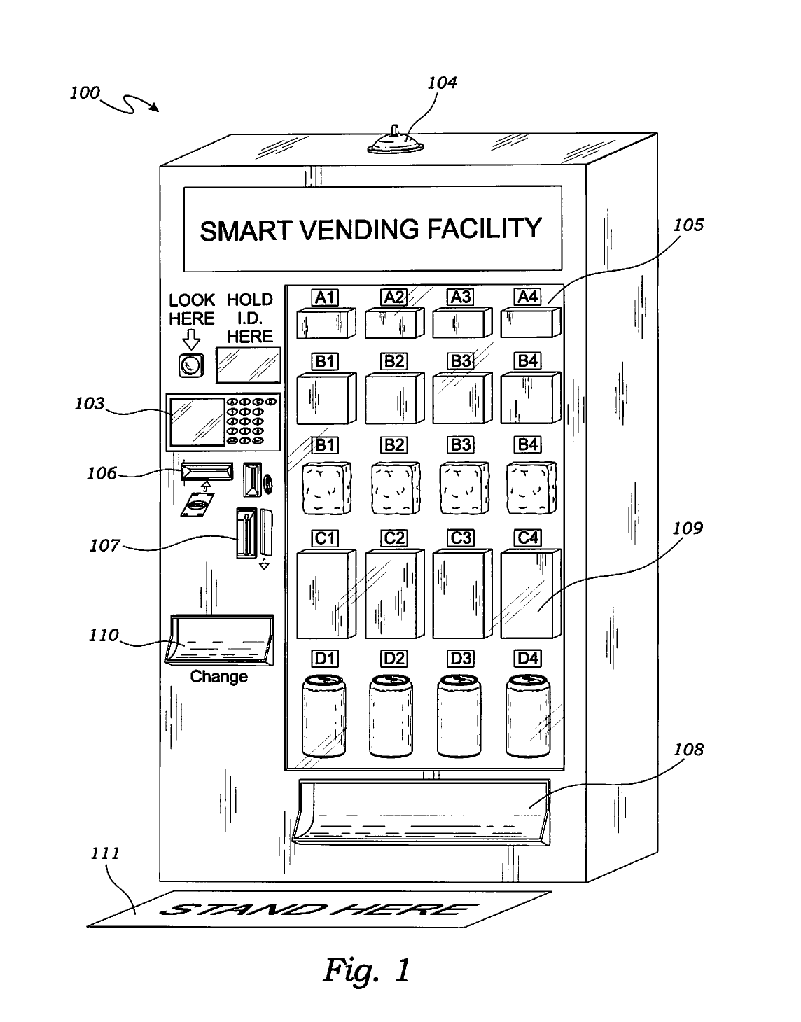

Described now in detail is the presently disclosed apparatus and method of use. FIG. 1 features an exemplar embodiment of a smart vending machine 100. Much like a traditional vending machine, the embodiment illustrated in FIG. 1 features a plurality of windows 109 through which a prospective consumer can view and select among the available regulated products. In the illustrated embodiment, each window 109 is assigned a unique alphanumeric designator 105 allowing for its selection via a push button sector and display 103. In some embodiments, the disclosed apparatus may be configured to feature fewer products or even a single product, and consequently, those embodiments may feature fewer windows 109 then illustrated in FIG. 1. Still other embodiments may feature no windows at all for various reasons including to reduce the visibility to children or individuals who do not approve of the vending of regulated products or for security purposes.

Further traditional features such as a features capable of accepting cash 106 and credit cards 107, dispensing change 110, and dispensing selected products 108 are also depicted. These features are necessary for the basic functions of a vending machine, namely product section, payment, and product dispensing and may appear in more advanced forms as technology is incorporated. For example, because a smart vending machine definitively identifies the prospective consumer, some embodiments may accept payment through invoicing or automatic charging of private club accounts or credit cards on file. Such embodiments may include no traditional features for accepting payment and dispensing change.

FIG. 1 also features an antenna 104 for wireless electromagnetic communication with an external database or the relevant permission granting authority. Some embodiments may communicate electromagnetically via a physical wire. The particular mode of communication is not an important to the present disclosure so long and the apparatus may electromagnetically communicate if the embodiment includes a remote database or requires communication with a permission granting authority.

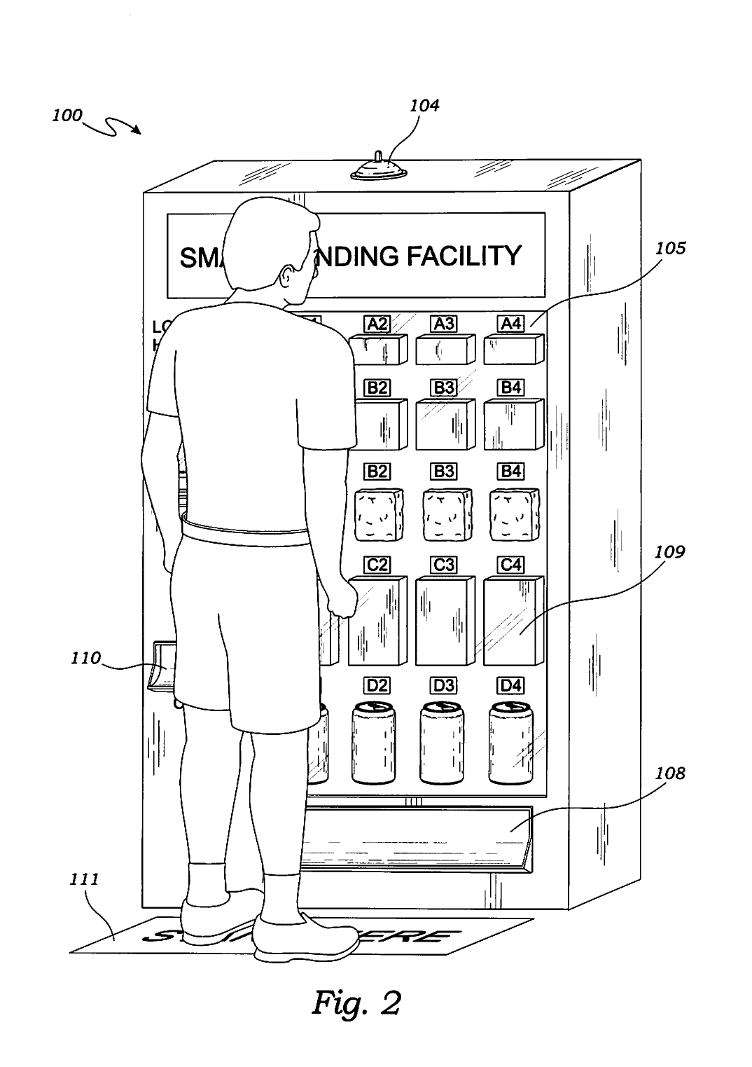

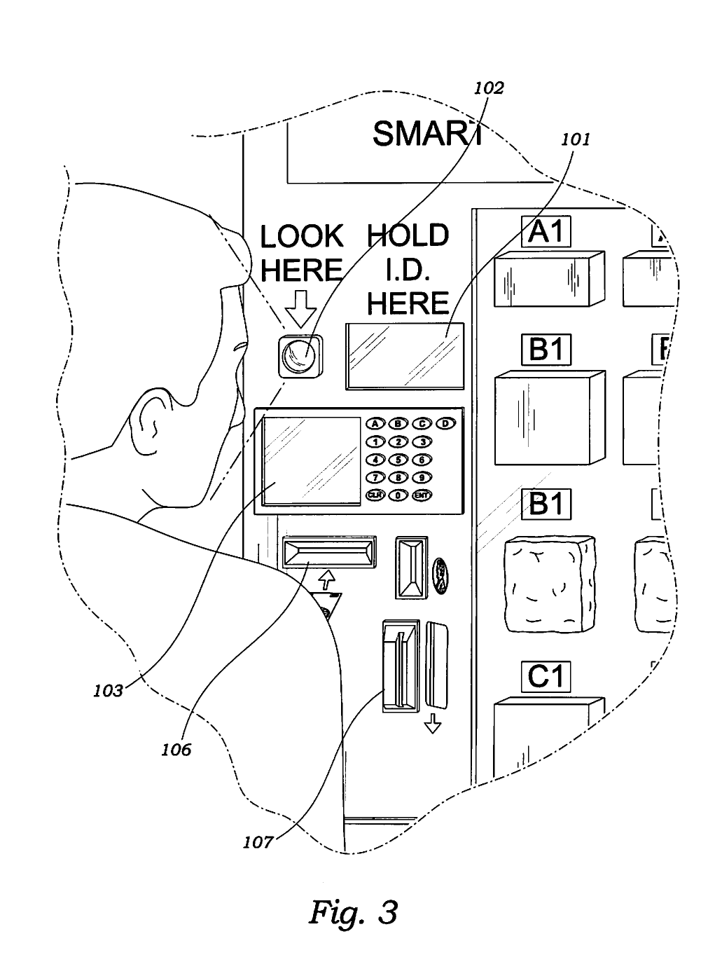

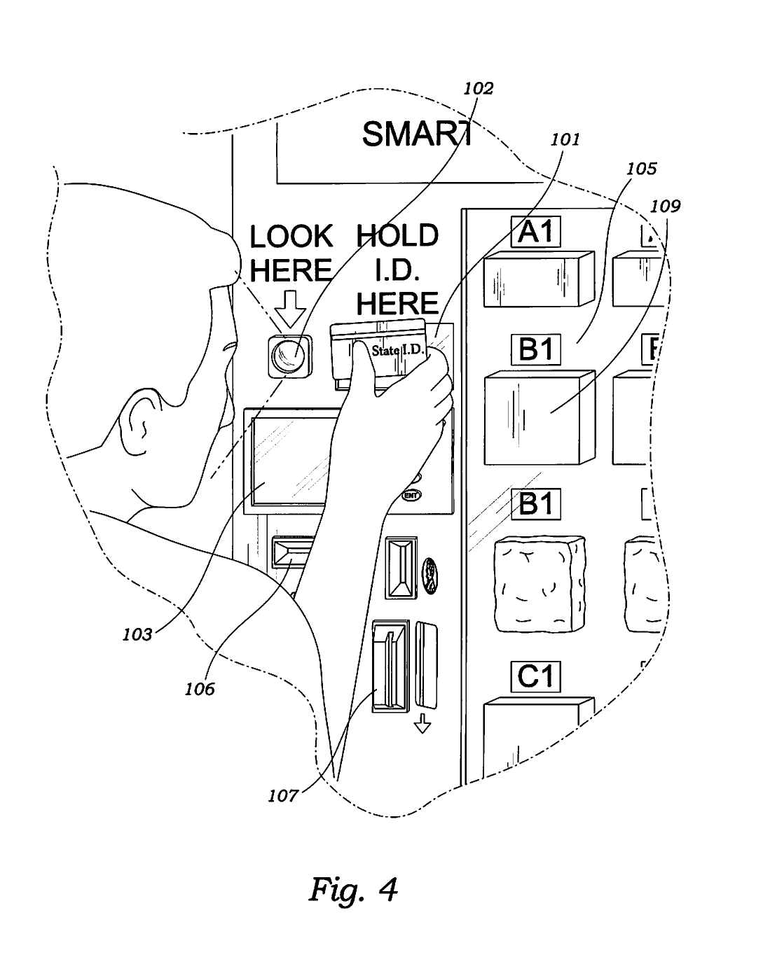

FIG. 2 illustrates a potential consumer standing before an illustrated embodiment preparing to begin the automated purchasing process. FIG. 3 illustrates a prospective customer engaging with exemplar identity verifying features. The embodiment depicts an identification scanner 101 and a biometric scanner 102. The illustrated identification scanner is capable of reading the unique identifying information from the identification when the prospective consumer holds it up to the scanner as illustrated in FIG. 4. This is just one of several forms of identification scanners that may be utilized. In some embodiments the identification scanner may require that the prospective customer swipe the identification or merely hold the identification in the immediate proximity so that the apparatus may scan the unique identifying information from the identification through RFID.

The biometric scanner featured in FIG. 4. is a camera capable of taking high quality pictures for identity verification through facial recognition software. A prospective customer is illustrated facing the camera so that unobstructed photographs may be taken for analysis. However, similar to the identification scanner, the illustrated biometric scanner is merely one acceptable example of identify verifying biometric scanners and should not be read as limiting. Other possible biometric scanners include a scale 111 to determine a prospective customer's weight as illustrated in FIG. 1, a height analysis determined by a camera, and/or a finger print, hand print and/or iris scanner. Some embodiments may utilize several biometric scanner and cross reference such results with the results of other scanners and pre-existing information in the database. This list is not intended to be limiting.

The enablement described in detail above is considered novel over the prior art of record and is considered to be critical to the operation of at least one aspect of the apparatus and its method of use, and to the achievement of the above-described objectives. The words used in this specification to describe the instant embodiments are to be understood not only in the sense of their commonly defined meanings, but to include by special definition in this specification: structure, material, or acts beyond the scope of the commonly defined meanings. Thus, if an element can be understood in the context of this specification as including more than one meaning, then its use must be understood as being generic to all possible meanings supported by the specification and by the word(s) describing the element.

The definitions of the words or drawing elements described herein are meant to include not only the combination of elements which are literally set forth, but all equivalent structures, materials or acts for performing substantially the same function in substantially the same way to obtain substantially the same result. In this sense it is therefore contemplated that an equivalent substitution of two or more elements may be made for any one of the elements described and its various embodiments or that a single element may be substituted for two or more elements in a claim.

Changes from the claimed subject matter as viewed by a person with ordinary skill in the art, now known or later devised, are expressly contemplated as being equivalents within the scope intended and its various embodiments. Therefore, substitutions, now or later known to one with ordinary skill in the art, are defined to be within the scope of the defined elements. This disclosure is thus meant to be understood to include what is specifically illustrated and described above, what is conceptually equivalent, what can be obviously substituted, and also what incorporates the essential ideas.

The scope of this description is to be interpreted only in conjunction with the appended claims and it is made clear, here, that the named inventors believe that the claimed subject matter is what is intended to be patented.

What is claimed is:

1. An apparatus for automatic vending of regulated products in compliance with applicable statutory regulations of the relevant jurisdiction, said apparatus comprising:
a vending machine configured to accept payment for and dispense a regulated product selected by a prospective consumer;
an identification scanner configured to scan information from a prospective consumer's identification;
a biometric scanner configured to scan at least one biometric quality of the prospective consumer;
a database with preexisting biometric and unique identifying information associated with the identification;
a programmable controller in electronic communication with said vending machine, said identification scanner, said biometric scanner, and said database wherein said programmable controller is programmed to verify the identity and eligibility of a prospective customer to purchase a particular regulated product and to control whether or not said vending machine proceeds to accept payment for and dispense said particular regulated product by comparing the biometric and unique identifying information associated with the identification contained in said database associated with the unique identifying information scanned from the proffered identification and the biometric information scanned directly from the prospective customer for congruency.

2. An apparatus of claim 1 wherein said database is a government owned and operated database located external to said vending machine with which said programmable controller is in electronic communication.

3. An apparatus of claim 1 wherein said database is a privately owned and operated database located external to said vending machine with which said programmable controller is in electronic communication.

4. An apparatus of claim 1 wherein said biometric scanner is a video image recorder capable of recording high quality images of the prospective consumer's facial features for the purpose of confirming the prospective consumer's identity by comparing such high quality recorded images to a preexisting recorded images located in said database associated with said identification using facial recognition software.

5. An apparatus of claim 1 wherein said biometric scanner is a skin image scanner capable of recording high quality images of the prospective consumers finger prints, palm prints, or hand prints for the purpose of confirming the prospective consumer's identity by comparing such high quality recorded images to a preexisting recorded images located in said database associated with said identification using pattern recognition software.

6. An apparatus of claim 1 wherein said biometric scanner is an iris scanner capable of recording high quality images of the prospective consumer's iris for the purpose of confirming the prospective consumer identity by comparing such high quality iris images to a preexisting recorded images located in said database associated with said identification using pattern recognition software.

7. An apparatus of claim 1 wherein said biometric scanner is a scale and camera capable of determining the prospective customer's height and weight for the purpose of confirming the prospective consumer identity by comparing such determined height and weight to a preexisting recorded metrics located in said database associated with said identification.

8. An apparatus of claim 1 further comprising a blood analyzer for the purpose of analyzing the chemical content of a prospective consumer to determine whether such prospective consumer is currently eligible to purchase the particular regulated product.

9. An apparatus of claim 8 wherein the blood analyzer is a breathalyzer capable of estimating the concentration of alcohol in the bloodstream of the prospective customer through an analysis of the prospective customer's breath.

10. An apparatus of claim 8 wherein said blood analyzer is a palm sweat analyzer capable of calculating the concentration of alcohol and/or other drugs in a prospective customer's bloodstream through an analysis of the prospective customer's palm sweat.

11. An apparatus of claim 1 further comprising:
an audio-video recorder configured to audio-visually record consumers while conducting transactions with said apparatus and store an audio-visual record of the transactions in said database; and
a tagging device configured to tag dispensed regulated products with a unique mark associated with the contemporary audio-visual record such that the audio-visual record of the transactions can be retroactively retrieved in said database and reviewed through use of said unique mark.

12. An apparatus of claim 1 wherein said database is in electronic communication with individualized permission granting authority such that the individualized permission granting authority may communicate individualized authorization to said database.

13. A method of automatically vending regulated products in compliance with applicable statutory regulations of the relevant jurisdiction, said apparatus comprising the steps of:
prompting a prospective consumer to select an available regulated product to purchase;
recording the prospective customer's selection;
prompting a prospective customer to present an appropriate form of identification;
scanning to record the unique identifying data contained on the identification;
comparing the scanned and recorded unique identifying data contained on the identification to preexisting unique identifying data contained in a database for identification validity verification;
calculating whether the perspective customer is eligible to purchase the selected regulated product based on the unique identifying data contained on the identification presented and the applicable statutory regulations of the relevant jurisdiction;
recording biometric information directly from the perspective customer;
comparing recorded biometric information with biometric information with preexisting biometric information to verify that the prospective customer is the individual to which the proffered identification was issued; and
prompting the prospective customer for payment if the proffered identification is verified to be valid, the application statutory regulation of the relevant jurisdiction allows the sale of the selected regulated product to the identified individual, and the prospective customer is verified to be the individual to whom the identification was issued;
dispensing the regulated product.

14. A method of claim 13 further comprising the steps of:
recording the time of day that the apparatus dispensed a regulated product to a particular individual in said database;
calculating how much time has elapse since the apparatus previously dispensed a regulated product to the same prospective customer; and
determining whether the time elapsed since the previous dispensing is temporally sufficient to authorize a subsequent dispensing.

15. A method of claim 13 further comprising the steps of:
recording quantity of regulated product that the apparatus dispensed within a particular time period to a particular individual in said database; and
determining whether the quantity of regulated product dispensed to a particular prospective customer within a particular time period is sufficiently low to authorize a subsequent dispensing.

16. A method of claim 13 further comprising the steps of:
prompting to the prospective customer to breath into a breathalyzer;
analyzing the prospective customer's current blood alcohol level; and
determining if the prospective customer's current blood alcohol level is sufficiently low to authorize a subsequent dispensing of a regulated product.

17. A method of claim 13 further comprising the steps of:
prompting the prospective customer to place an hand of a palm sweat analyzer;
analyzing the prospective customer's palm sweat; and
calculating whether the prospective customer's palm sweat is sufficiently free of drugs to authorize subsequent dispensing of a regulated product.

18. A method of claim 13 further comprising the steps of:
prompting the prospective customer to cooperate with a blood concentration analyzer;
analyzing the prospective customer's results; and
calculating whether the prospective customer's results are sufficient to authorize subsequent dispensing of a regulated product.

19. A method of claim 13 further comprising the steps of:
receiving notice of individuals who have received individualized permission to purchase a particular regulated product from the individualized permission granting authority;
storing a record of the individuals who have received individualized permission from the individualized permission granting authority in said database;
determining whether a prospective customer is an individual who has received individualized permission; and
dispensing of the particular regulated product if authorized.

20. A method of claim 13 further comprising the steps of:
audio-visually recording a prospective customer;
tagging dispensed regulated product with a unique mark;
storing the audio-visual recording in database associated with unique mark; and
retrieving audio-visual recording upon request via unique mark.

21. A method of claim 13 further comprising the steps of:
registering prospective customers to be eligible to purchase regulated products through said apparatus at a prior time and place;
recording the prospective customer's biometric information and storing it in the database the time of registration;
recording the prospective customer's unique identifying information contained on the prospective customer's identification and storing it in the database at the time of registration; and
associating the prospective customer's recorded biometric information and recorded unique identifying information contained on the prospective customer's identification in the database at the time of registration.
